(19) United States
(12) Patent Application Publication
(10) Pub. No.: US 2024/0197201 A1
(43) Pub. Date: Jun. 20, 2024

(54) Remote Monitoring of Respiration

(71) Applicant: University College Dublin, National University of Ireland, Dublin 4 (IE)

(72) Inventors: Emer Doheny, Blackrock Co., Dublin (IE); Madeleine Lowery, Dublin 3, Co. Dublin (IE); Ben O'Callaghan, Kilmacanogue Co., Wicklow (IE)

(21) Appl. No.: 18/573,903

(22) PCT Filed: Jun. 23, 2022
(86) PCT No.: PCT/EP2022/067119

§ 371 Date: Dec. 22, 2023

(30) Foreign Application Priority Data
Jun. 24, 2021 (GB) 2109116.0

(51) Int. Cl. A61B 5/08; A61B 5/00; G16H 20/40

(52) U.S. Cl. CPC A61B 5/0816; A61B 5/0002; G16H 20/40

(57) ABSTRACT

A computer-implemented method is provided for characterising breathing audio data. The method/system comprises acquiring breathing audio data. The method/system further comprises determining an estimated respiration rate based on the breathing audio data. The method/system also comprises identifying exhales in the breathing audio data using the estimated respiration rate. One or more computer-readable storage media are also provided comprising instructions that, when executed by a computer, cause the computer to determine an estimated respiration rate based on breathing audio data, and identify exhales in the breathing audio data using the estimated respiration rate.

### FIELD OF THE INVENTION

The invention relates to the field of remote monitoring of respiration, for example using smartphones or other smart devices.

### BACKGROUND

Respiration rate is an important vital sign, and respiratory distress is a characteristic symptom of COVID-19, with most patients admitted to intensive care unable to breathe spontaneously. The most common reason for a patient to require intensive care has been respiratory support, with increased respiratory rate observed in these patients, and two-thirds experiencing acute respiratory distress syndrome. Dyspnoea has been shown to be a persistent symptom in patients with acute COVID-19. Even among individuals with moderate symptoms, respiration rate has been shown to be elevated following symptom onset with a slow return to baseline values.

Remote monitoring of patient symptoms during COVID-19 and other respiratory diseases is desirable as this obviates the need for patients to visit a clinical setting or to leave their own homes. Remote monitoring is particularly beneficial in pandemic situations such as the COVID-19 pandemic due to the need for affected individuals to self-isolate and the overwhelming demand on the healthcare system during the pandemic. However, when monitoring respiratory symptoms, most remote monitoring systems rely on self-reported measures of dyspnoea, or shortness of breath. Monitoring respiration using smartphones or other portable smart devices would allow individuals to accurately monitor their own respiratory health, and clinicians to remotely monitor large cohorts of patients in their own homes. This would be particularly valuable in monitoring COVID-19 patients in the early stages of the disease, or after discharge from hospital. Such a monitoring tool would also have wide applicability in the management of chronic respiratory diseases.

Remote data collection using smartphones poses challenges for data quality and consistency. The signal quality of audio data recorded remotely using a smartphone may be influenced by a range of hardware and software factors, such as the technical specifications and location of the microphone, the sampling rate of the audio data, the software application used to record data, and the operating system of the phone. Human factors may also influence remote audio recordings of breathing, such as environmental noise or inter-subject variations in signal amplitude or interpretation of a protocol. Inhalation is particularly difficult to record as it is often barely audible.

There remains a need to develop a reliable and low-cost method for unsupervised remote monitoring of respiration that can be used by large numbers of individuals with both healthy and pathological respiration patterns in a wide variety of settings using non-clinical hardware that is widely available. There is also a need for methods for remotely monitoring a wider variety of respiration parameters other than the respiration rate, such as exhalation duration.

### SUMMARY OF THE INVENTION

The invention provides a computer-implemented method for characterising breathing audio data. The method comprises acquiring breathing audio data. The method further comprises determining an estimated respiration rate based on the breathing audio data, and identifying exhales in the breathing audio data using the estimated respiration rate.

The breathing audio data generally represent, e.g. encode, a breathing audio signal. The estimated respiration rate may therefore be an estimated respiration rate of the breathing audio signal. Likewise, identifying the exhales in the breathing audio data may comprise identifying exhales in the breathing audio signal that the breathing audio data represent.

The method may further comprise determining a refined respiration rate based on the identified exhales.

Determining the estimated respiration rate may comprise performing a spectral analysis of the breathing audio data to determine the estimated respiration rate.

Determining the estimated respiration rate may comprise calculating a frequency spectrum of the breathing audio data, and determining the estimated respiration rate based on the frequency spectrum. The frequency spectrum may be a power spectrum of the breathing audio data.

Calculating the frequency spectrum may comprise calculating a first audio signal envelope of the breathing audio data. The frequency spectrum may be a frequency spectrum of the first audio signal envelope.

Calculating the first audio signal envelope may comprise rectifying the breathing audio data/, and/or low-pass filtering the breathing audio data.

The method may comprise determining a fundamental frequency of the breathing audio data based on the frequency spectrum. The fundamental frequency may be the fundamental frequency of the first audio signal envelope. The estimated respiration rate may be determined based on the fundamental frequency.

The method may comprise calculating a harmonic product spectrum of the breathing audio data (e.g. of the first audio signal envelope) based on the frequency spectrum (e.g. the power spectrum), and identifying the fundamental frequency based on the harmonic product spectrum.

The frequency spectrum may be determined using a method of averaged periodograms. The method of averaged periodograms may be Welch's method or Bartlett's method.

The frequency spectrum may be determined using a window function having an adaptable length. Calculating the frequency spectrum may comprise determining whether the breathing audio data contains anomalous features, and adapting the length of the window function based on whether the breathing audio data is determined to contain anomalous features.

The length of the window function may be reduced if the breathing audio data is determined to contain anomalous features.

The length of the window function may be adapted to a first length if the breathing audio data is determined to not contain anomalous features, or to a second length if the breathing audio data is determined to contain anomalous features, wherein the second length is shorter than the first length.

Optionally, the breathing audio data is determined to contain anomalous features if one or more large peaks having an amplitude exceeding a threshold amplitude is identified in the breathing audio data, e.g. in the first audio signal envelope. In other words, the anomalous features may comprise large peaks, such as those having an amplitude exceeding a threshold amplitude.

The large peaks may be rescaled to reduce their amplitude in the breathing audio data prior to calculating the frequency spectrum.

Identifying the exhales in the breathing audio data using the estimated respiration rate may comprise calculating a second audio signal envelope of the breathing audio data. The second audio signal envelope may be the same as the first audio signal envelope.

Identifying the exhales in the breathing audio data using the estimated respiration rate may comprise identifying exhales in the breathing audio data using an exhale identification algorithm adapted based on the estimated respiration rate.

The exhale identification algorithm may employ an adaptive thresholding method that is adapted based on the estimated respiration rate. The adaptive thresholding method may be applied to the breathing audio data, for example it may be applied to the second audio signal envelope.

The length of a moving window function employed by the adaptive thresholding method may be adapted based on the estimated respiration rate. The moving window function may be employed to determine an adaptive threshold used to identify the exhales.

A degree of overlap of the moving window function may be adapted based on the estimated respiration rate.

The method may further comprise calculating an expected inter-breath period based on the respiration rate. The length and/or degree of overlap of the moving window function employed by the adaptive thresholding method may adjusted based on the expected inter-breath period.

The estimated respiration rate may be used to identify exhales missed by the exhale identification algorithm and/or to identify spurious exhales identified by the exhale identification algorithm.

Optionally, adjacent exhales identified by the exhale identification algorithm are merged if an inter-breath period between them is shorter than a minimum inter-breath period threshold. The minimum inter-breath period threshold may be determined based on the estimated respiration rate.

Optionally, missed exhales are searched for between adjacent exhales identified by the exhale identification algorithm that are separated by an inter-breath period that exceeds a maximum inter-breath period threshold. The maximum inter-breath period threshold may be determined based on the estimated respiration rate.

Optionally, exhales identified by the exhale identification algorithm having a duration longer than a maximum exhale duration threshold are discarded. The maximum exhale duration threshold may be determined based on the estimated respiration rate.

Optionally, if the interval separating adjacent exhales is shorter than a minimum interval threshold, the shorter of the adjacent exhales is discarded. The minimum interval threshold may be determined based on the estimated respiration rate.

The method may further comprise classifying the quality of the breathing audio data as acceptable or unacceptable for identifying exhales using a signal classifier trained by machine learning to classify the quality of breathing audio data as acceptable or unacceptable for identifying exhales. The steps of identifying exhales in the breathing audio data using the estimated respiration rate and determining a refined respiration rate based on the identified exhales may be performed only if the quality of the audio data is classified as acceptable.

The method may further comprise, if the quality of the audio data is classified as unacceptable, issuing an instruction that the breathing audio data must be re-recorded. The method may further comprise acquiring re-recorded breathing audio data.

The signal classifier may have been trained using a training dataset comprising a plurality of breathing audio data recordings previously classified as being acceptable or unacceptable for identifying exhales. For example, the breathing audio data recordings previously classified as being acceptable or unacceptable for identifying exhales may have been classified by one or more persons or individuals.

The signal classifier may employ the estimated respiration rate to determine whether the quality of the breathing audio data is acceptable or unacceptable for identifying the exhales. In other words, the signal classified may determine whether the quality of the breathing audio data is acceptable or unacceptable for identifying the exhales based, at least in part, on the estimated respiration rate.

The invention also provides a computer system for characterising breathing audio data. The computer system is configured to: acquire breathing audio data; determine an estimated respiration rate based on the breathing audio data; and identify exhales in the breathing audio data using the estimated respiration rate.

The invention further provides one or more computer-readable storage media comprising instructions that, when executed by a computer, cause the computer to: determine an estimated respiration rate based on breathing audio data; and identify exhales in the breathing audio data using the estimated respiration rate.

The invention also provides a computer-implemented method for classifying the quality of breathing audio data as acceptable or unacceptable for use in determining or identifying one or more respiration features. The method comprises: acquiring breathing audio data; classifying the quality of the breathing audio data as acceptable or unacceptable for determining the one or more respiration features using a signal classifier trained by machine learning to classify the quality of breathing audio data as acceptable or unacceptable for determining or identifying the one or more respiration features.

The invention also provides a computer system for classifying the quality of breathing audio data as acceptable or unacceptable for use in determining or identifying one or more respiration features, the computer system is configured to classify the quality of the audio data as acceptable or unacceptable for determining the one or more respiration features using a signal classifier trained by machine learning to classify the quality of breathing audio data as acceptable or unacceptable for determining or identifying the one or more respiration features. For example, the computer system may comprise a processor configured to classify the quality of the audio data as acceptable or unacceptable for determining the one or more respiration features using a signal classifier trained by machine learning to classify the quality of breathing audio data as acceptable or unacceptable for determining or identifying the one or more respiration features. The computer system may comprise one or more computer-readable storage media comprising (i.e. storing or having loaded thereon) instructions that, when executed by the processor, cause the processor to: classify the quality of breathing audio data as acceptable or unacceptable for determining or identifying one or more respiration features using a signal classifier trained by machine learning to classify the quality of breathing audio data as acceptable or unacceptable for determining or identifying the one or more respiration features. The processor may be configured to execute the instructions stored on the computer-readable storage media to classify the quality of breathing audio data as acceptable or unacceptable for determining or identifying the one or more respiration features.

The invention also provides one or more computer-readable storage media comprising instructions that, when executed by a computer, cause the computer to: classify the quality of breathing audio data as acceptable or unacceptable for determining or identifying one or more respiration features using a signal classifier trained by machine learning to classify the quality of breathing audio data as acceptable or unacceptable for determining or identifying the one or more respiration features.

### BRIEF DESCRIPTION OF THE FIGURES

The invention will now be described, by way of example only, with reference to the appended drawings.

FIG. 1 is a flow diagram illustrating a method for characterising breathing audio data in accordance with the invention.

FIG. 2 shows an example of breathing audio data of the sort used by the methods of the invention.

FIG. 3 is a flow diagram illustrating a method for characterising breathing audio data in accordance with the invention.

FIG. 4 is a flow diagram illustrating a method for classifying the quality of breathing audio data in accordance with the invention.

FIG. 5a shows raw breathing audio data.

FIG. 5b shows a power spectrum of the breathing audio data shown in FIG. 5a.

FIG. 6 is a table summarising the features employed by the machine learning algorithm used to train the signal classifier of the example embodiment.

FIG. 7a shows an example of raw breathing audio data with exhales and inhales annotated.

FIG. 7b shows an envelope of the audio data shown in FIG. 7a obtained by signal rectification and low-pass filtering.

FIG. 7c shows the envelope shown in FIG. 7b after smoothing with an adaptive threshold based on Otsu's thresholding technique overlaid.

FIG. 7b shows exhales identified using the adaptive threshold as a square wave function (binary activity).

FIG. 8 shows an example signal envelope of breathing audio data, an adaptive threshold based on Otsu's image thresholding technique, and exhales identified using the adaptive threshold.

FIG. 9 is a table summarising the features employed by the signal classifier of the example embodiment, the feature importance, and the values of the features (mean±standard deviation) for all acceptable and unacceptable recordings.

FIG. 10a is a boxplot of the respiration rate (bpm) for the three subject groups (controls, COVID day >21 and COVID day 1-21) of the example calculated by the algorithm of the example embodiment (solid fill boxes, left) and from the researcher annotations (hatched boxes, right). All 192 acceptable recordings are included.

FIG. 10b is a boxplot of the exhale duration (s) for the three subject groups (controls, COVID day >21 and COVID day 1-21) of the example calculated by the algorithm of the example embodiment (solid fill boxes, left) and from the researcher annotations (hatched boxes, right). All 192 acceptable recordings are included.

FIG. 10c is a boxplot of the normalized exhale duration (%) for the three subject groups (controls, COVID day >21 and COVID day 1-21) of the example calculated by the algorithm of the example embodiment (solid fill boxes, left) and from the researcher annotations (hatched boxes, right). All 192 acceptable recordings are included.

### DETAILED DESCRIPTION

The following description is intended to introduce various aspects and features of the invention in a non-limiting manner. For clarity and brevity, features and aspects of the invention may be described in the context of particular embodiments. However, it should be understood that features of the invention that are described only in the context of one or more embodiments may be employed in the invention in the absence of other features of those embodiments, particularly where there is no inextricable functional interaction between those features. Even where some functional interaction between the features of an embodiment is discernible, it is to be understood that those features are not inextricably linked if the embodiment would still fulfil the requirements of the invention without one or more of those features being present. Thus, where features are, for brevity, described in the context of a single embodiment, those features may also be provided separately or in any suitable sub-combination. It should also be noted that features that are described in the context of separate aspects and embodiments of the invention may be used together and/or be interchangeable wherever possible. Features described in connection with the invention in different contexts (e.g. a method, system, computer readable medium/media and/or computer program) may each have corresponding features definable and/or combinable with respect to each other, and these embodiments are specifically envisaged.

The invention provides an improved means for characterising breathing audio data, in particular breathing audio data recoded using a personal smart device such as a smartphone, tablet, or other smart device. For example, the invention provides a means for determining or identifying respiration features, such as exhales and/or a respiration rate, from breathing audio data. As such, the invention provides a method for remote monitoring of respiration. The method of the invention involves determining an estimated respiration rate based on breathing audio data, and then identifying individual exhales in the breathing audio data using, i.e. based on, the estimated respiration rate to improve the accuracy of exhale identification. A refined respiration rate may then be determined based on the identified exhales, for example by counting the identified exhales.

Using the estimated respiration rate to guide the identification of the exhales results in the exhales being more accurately identified, with fewer exhales missed and fewer spurious exhales identified. In other words, using the estimated respiration rate to identify the individual exhales in the breathing audio data refines the identification of the exhales and therefore provides a more reliable output. The refined respiration rate that is calculated based on the identified individual exhales is therefore more accurate than the initial estimated respiration rate determined from the breathing audio data before the individual exhales are identified.

Determining the respiration rate based on individual exhales, for example based on the number of identified exhales, also provides a more accurate means of determining the respiration rate than, for example, performing a spectral analysis of the breathing audio data. Audio spectral analysis methods assume a regular and predictable breathing pattern, and deviations from such idealised breathing patterns can cause problems for such methods. On the other hand, the method of the invention, which calculates the respiration rate based on identified individual exhales, can cope with irregular inter-breath periods and irregular exhale amplitudes, which generally cause problems for the accuracy of spectral analysis methods.

A further advantage of reliably and accurately identifying the individual exhales is that further respiration parameters can be calculated, such as average exhale duration, inter-breath period variation (e.g. variance or standard deviation), a ratio of average exhale duration to respiration rate, or a ratio of average exhale duration to average full breath duration (i.e. a ratio of average exhale duration to average inter-breath period). Where the word “average” is used here, it is generally used to mean the arithmetic mean, but other averages could also be employed. These exhale-derived respiration parameters provide further useful insight into patient health, and may be a valuable tool to the clinician. For example, the ratio of exhale duration to inter-breath period may be helpful in identifying patients that are deteriorating and in need of medical intervention.

The breathing audio data are audio data that record the breathing sounds, or respiration sounds, of an individual, i.e. a person. The breathing sounds may be nasal and/or tracheal sounds. The breathing audio data therefore represent or characterise, e.g. encode, a breathing or respiration audio signal of an individual, and the terms “audio signal” and “audio data” are therefore used interchangeably herein where referring to the processing of the audio data. The breathing audio data, sometimes alternatively referred to as respiration audio data, are generally recorded by a microphone. The method of the invention is particularly well-suited to characterising breathing audio data recorded by a portable smart device, such as a smartphone, tablet computer, or other portable smart device, due to the robustness of the signal processing method and the ability of the processing method to handle irregular breathing patterns and signal artefacts that more commonly affect recordings made using such devices. However, other audio recording devices could be used to record the breathing audio data.

The methods of the invention are implemented on a computing system and are therefore computer-implemented methods. For example, the method may be performed by the processor of a computing system, which executes instructions stored on one or more computer-readable storage media. The method steps are therefore performed by a computer. The computer system may therefore comprise one or more computer-readable storage media that store instructions for performing a method in accordance with the invention, and one or more processors that are configured to execute the instructions to perform a method in accordance with the invention. The computing system on which the methods of the invention are implemented may be the same device as that used to record the audio data, or it may be different. For example, the audio data may be recorded by a remote device, such as a smartphone or other personal smart device, and the audio data may be transferred or uploaded to the computing system that performs the methods of the invention. In particular, the computing system that performs the methods of the invention may be a server computer to which the audio data are uploaded, for example wirelessly over a network, once they have been recorded. As used herein, the terms “computing system” and “computer” are used interchangeably, and should be understood to encompass either a single device or a distributed system.

Each method disclosed herein has associated with it a corresponding computer system configured to perform the method, and a corresponding one or more computer-readable storage media comprising (i.e. having stored thereon) instructions that, when executed by a computer, cause the computer to perform the method, and these systems and method are specifically disclosed.

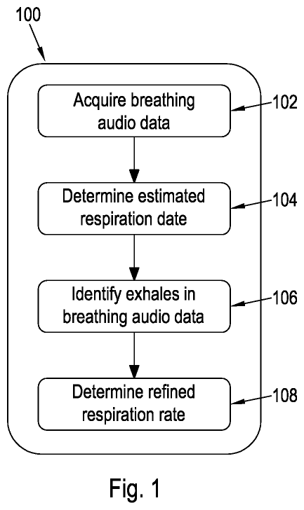

Referring to FIG. 1, a method 100 for characterising breathing audio data may comprise step 102 of acquiring breathing audio data. For example, the computer that performs the method may acquire the breathing audio data and may store the breathing audio data in one or more computer-readable media. Data acquisition step 102 may comprise recording the breathing audio data, or it may comprise receiving breathing audio data that has been previously recorded.

The method 100 comprises step 104 of determining an estimated respiration rate based on, or using, the breathing audio data. In other words, method 100 comprises processing the breathing audio data to determine the estimated respiration rate of the audio signal. The estimated respiration rate may be determined by performing a spectral analysis of the breathing audio data. Audio spectral analysis methods process an audio signal to yield a frequency spectrum, for example the power spectrum, of the audio signal. In the context of the invention, a spectral analysis of the breathing audio signal may be performed to generate a frequency spectrum of the breathing audio signal, and the estimated respiration rate may be determined based on the frequency spectrum. For example, the estimated respiration rate may be based on the frequency of a peak in the frequency spectrum, namely the frequency of the peak that corresponds to the fundamental frequency of the audio signal, or the dominant (i.e. largest amplitude) peak in the power spectrum, which generally corresponds to the fundamental frequency. In step 104 the estimated respiration rate may be determined directly from the raw breathing audio signal, or the breathing audio signal may be pre-processed to varying degrees before the estimated respiration rate is determined. For example, the audio signal may be rectified and an envelope of the audio signal may be calculated, and the estimated respiration rate may be determined based on the audio signal envelope, for example by performing a spectral analysis of the audio signal envelope.

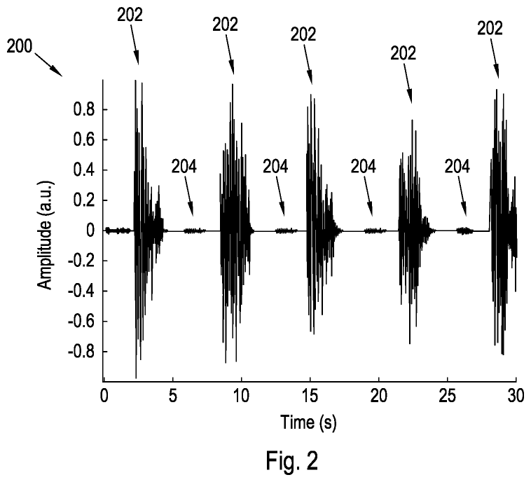

The method 100 further comprises step 106 of identifying individual exhales, or exhalations, in the breathing audio data using the estimated respiration rate determined in step 104. FIG. 2 shows a typical audio signal 200, with the exhales 202 and inhales 204 identified. As can be seen from FIG. 2, the inhales have a substantially larger amplitude than the inhales. Identifying the exhales is therefore a reliable way of identifying each breath in the audio data because of the relative strength of the exhale signal and the good separation of each exhale by a relatively low-amplitude inhale. Using the estimated respiration rate to identify the exhales improves the accuracy and reliability with which the exhales are identified. This is because the expected separation between the exhales is related to the respiration rate, and using the respiration rate to inform the algorithm that is used to identify the exhales therefore improves the accuracy of the algorithm.

Step 106 may involve using the respiration rate directly to identify the exhales, or may involve using a respiration parameter derived from or based on the estimated respiration rate, such as an expected or average (e.g. mean) inter-breath period, where the inter-breath period is the period between the same point in successive breaths, for example the period between the onsets of successive exhalations, as the term period is commonly understood to mean in the context of periodic and repeating signals. The expected inter-breath period may also be referred to as the expected or average respiration period. The expected inter-breath period is a reciprocal of the estimated respiration rate. For example, where the estimated respiration rate is given in units of breaths per minute the inter-breath period in seconds is given by 60/estRR, where estRR is the estimated respiration rate. Where a parameter derived from the estimated respiration rate is used to optimise the identification of the exhales this is still considered to be a use of the estimated respiration rate in this context because the respiration parameter is calculated based on the respiration rate and the identification of the exhales is therefore still performed using the estimated respiration rate, namely to determine the respiration parameters.

The exhales may be identified using an algorithm, and the algorithm may be adapted or optimised based on the value of the estimated respiration rate. For example, the estimated respiration rate, or a respiration parameter based on the estimated respiration rate, may be used as an input parameter to the algorithm and the algorithm may search for exhales using the input parameter.

The method may further comprise step 108 of determining a refined respiration rate based on the identified exhales. For example, the refined respiration rate may be determined based on the number of exhales identified in the audio signal. This is because each exhale corresponds to one breath, or respiration cycle, and the number of exhales per unit time is equivalent to the respiration rate. The refined respiration rate may therefore be calculated as the number of exhales identified per unit of time.

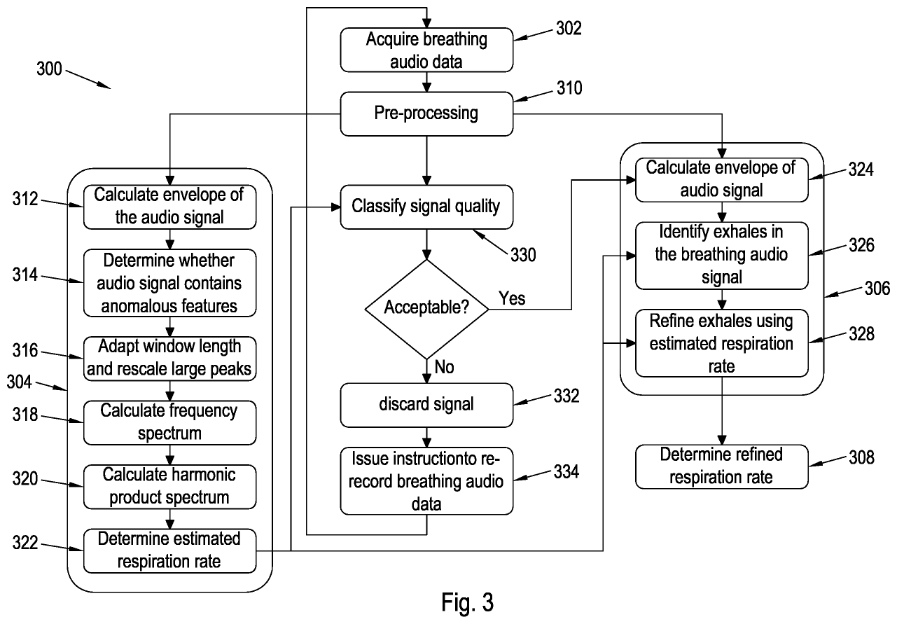

FIG. 3 illustrates a more specific implementation of the method 300 of the invention, illustrating various additional optional steps. It should be noted that although the various steps illustrated in FIG. 3 are shown in a single scheme these additional method steps could be employed separately and distinctly of one another within the context of the invention.

After step 302 of acquiring the breathing audio data, the audio data may be pre-processed in step 310. For example, the audio signal may be low-pass filtered. Low-pass filtering may be performed using a Butterworth filter. The pre-processing may comprise clipping the breathing audio data to remove start and end portions of the audio signal so as to reduce signal artefacts. The clipped audio data may be used in steps 304 and 330, but step 306 may use the entire (unclipped) recording.

Step 304 of determining an estimated respiration rate based on the breathing audio data may comprise step 312 of calculating an envelope of the breathing audio signal. Calculating the signal envelope may comprise rectifying the breathing audio data, and/or low-pass filtering the breathing audio data. Low-pass filtering may be performed using a Butterworth filter. A notch filter may also be implemented to remove electrical noise from the breathing audio data. For example, a notch filter may be applied to the breathing audio data to remove 50 Hz electrical noise that may be present if the device used to record the audio data, e.g. a smartphone, is plugged in and is charging during the recording. If a signal envelope is calculated in step 312 the subsequent processing steps are performed on the signal envelope, and references to the audio signal or audio data may be taken to mean the audio signal envelope.

Step 304 may comprise step 318 of calculating a frequency spectrum of the breathing audio signal, for example a frequency spectrum of the audio signal envelope. In particular, step 318 may comprise calculating a power spectrum of the breathing audio signal. The estimated respiration rate may be determined based on the frequency spectrum. For example, step 322 of determining the estimated respiration rate may comprise determining the fundamental frequency of the breathing audio data (e.g. the fundamental frequency of the signal envelope) based on the frequency spectrum, and the respiration rate may be determined based on the fundamental frequency. In particular, the fundamental frequency may be used as the estimated respiration rate. The fundamental frequency of the audio signal may be determined by performing a peak analysis of the frequency spectrum. For example, a peak in the spectrum may be identified as corresponding to the fundamental frequency, and the frequency of that peak may be used as the estimated respiration rate.

The power spectrum may be determined using a method of averaged periodograms, sometimes called periodogram methods. For example, the power spectrum may be calculated using Welch's method or Bartlett's method, in particular Welch's method. Both Welch's method and Bartlett's method apply a moving window function to the audio signal, thereby dividing the signal into segments. In Welch's method these segments overlap, whereas in Bartlett's method they are non-overlapping. The window function may be a Hamming window, for example.

In order to improve the calculation of the frequency spectrum in step 318 and to reduce adverse effects caused by unusual or anomalous features in the audio signal, such as unusual breathing patterns or signal artefacts, steps 314 and 316 may be performed prior to step 318. Self-recorded audio data that are recorded using personal devices such as smartphones or the like are susceptible to the adverse effects of signal artefacts, such as might be caused by a change of phone position or background noise, and unsupervised recordings often contain unusual breathing patterns such as coughing, sighing or yawning. Steps 314 and 316 serve to mitigate the effects of these anomalous features and therefore improve the reliability and accuracy of the respiration rate estimate.

Step 314 of determining whether the breathing audio signal contains anomalous features may comprises identifying peaks in the breathing audio signal (e.g. the signal envelope), and determining that the breathing audio signal contains anomalous features if one or more large peaks having an amplitude exceeding a threshold amplitude is identified in the breathing audio data. In one example implementation, the largest peak in the signal (i.e. having the largest amplitude) is identified, and the breathing audio signal may be determined to contain anomalous features if the largest peak has an amplitude exceeding a threshold amplitude. The threshold amplitude may be determined based on, or relative to, an average amplitude of all of the peaks identified in the breathing audio data, such as the median amplitude of all of the peaks identified in the breathing audio data. For example, the threshold amplitude may be set in proportion to an average amplitude of all of the identified peaks, in particular it may be proportional to the median amplitude of the identified peaks. In one particular example the threshold amplitude may be a multiple of the median amplitude, such as five times the median amplitude.

The window function employed to determine the power spectrum may have an adaptable length, and the length of the window function may be adapted in step 316 based on whether the breathing audio signal is determined to contain anomalous features, for example whether the breathing audio signal is determined to contain large peaks having an amplitude that exceeds a threshold amplitude. In particular, the length of the window function may be reduced if the breathing audio data is determined to contain anomalous features. For example, the window function may have a default length, and the reduced length may be used if the breathing audio data is determined to contain anomalous features. In other words, the length of the window function may be set to a first, longer, length if the breathing audio data is determined to not contain anomalous features or to a second, shorter, length if the breathing audio data is determined to contain anomalous features. Reducing the length of the window function in this way mitigates for the effects of the anomalous features (e.g. large peaks) in the signal and provides improved results for audio signals that are affected by such unusual features.

Separately, step 316 may comprise rescaling any large peaks that are identified in the audio signal to reduce their amplitude prior to calculating the power spectrum of the breathing audio data in step 318. For example, the large peaks may be rescaled to a lower amplitude determined based on an average amplitude of all of the peaks identified in the audio signal. In one particular example, the amplitude of the large peaks may be reduced to be equal to the median amplitude of all of the peaks identified in the audio signal. Reducing the amplitude of the large peaks in this way mitigates against the otherwise negative effects of such large peaks on the calculation of the power spectrum and therefore improves the reliability and accuracy of the respiration rate estimate.

In other words, determining the estimated respiration rate based on the breathing audio data may comprise identifying any large peaks in the breathing audio data (e.g. in the signal envelope) having an amplitude exceeding a threshold amplitude, rescaling the large peaks to reduce their amplitude in the breathing audio data, and then determining the estimated respiration rate based on the breathing audio data containing the rescaled large peaks.

Step 304 of determining the estimated respiration rate based on the breathing audio data may further comprise step 320 of calculating a harmonic product spectrum of the breathing audio data, for example the harmonic product spectrum of the signal envelope. The harmonic product spectrum is calculated based on the power spectrum, i.e. the power spectrum is used to generate the harmonic product spectrum. The estimated respiration rate may then be determined based on the harmonic product spectrum. In particular, the fundamental frequency of the breathing audio signal may be determined from the harmonic product spectrum. Employing the harmonic product spectrum to determine the fundamental frequency of the audio signal improves the reliability of correctly identifying the fundamental frequency rather than a harmonic, and therefore improves the reliability and accuracy of the respiration rate estimate.

The lowest-frequency peak in the harmonic product spectrum that has an amplitude that exceeds a threshold amplitude and/or a frequency that exceeds a minimum acceptable estimated respiration rate may be identified as the fundamental frequency. The threshold amplitude may be determined based on the amplitude of the largest peak in the harmonic product spectrum. For example, the threshold amplitude may be set relative to, or in proportion to, the amplitude of the largest peak. In particular, the threshold value may be set to be equal to a predetermined proportion of the amplitude of the largest peak in the harmonic product spectrum, such as 80%. These conditions help to ensure that the actual fundamental frequency is identified accurately.

Step 306 of identifying exhales in the breathing audio data using the estimated respiration rate may comprise step 324 of calculating an envelope of the breathing audio signal. Calculating the signal envelope may comprise rectifying the breathing audio data, and/or low-pass filtering the breathing audio data. Low-pass filtering may be performed using a Butterworth filter. The resulting signal envelope may also be smoothed. The envelope may be the same or different to the signal envelope calculated in step 312. If a signal envelope is calculated in step 324 the subsequent processing steps are performed on the signal envelope, and references to the audio signal or audio data may be taken to mean the audio signal envelope calculated in step 324.

Step 306 of identifying exhales in the breathing audio data using the estimated respiration rate may comprise step 326 of identifying individual exhales in the audio signal. The individual exhales may be identified using an exhale identification algorithm, which may employ a thresholding method in which a threshold is applied to the audio signal envelope to identify the exhales. In particular, individual exhales may be identified as portions of the audio signal envelope that exceed the threshold. Alternatively, the exhale identification algorithm may employ other methods to identify the exhales, such as a peak detection method, and peaks in the audio signal envelope that exceed a threshold, for example an adaptive threshold, may be identified as exhales, with the locations of the local minima in the signal envelope either side of each identified exhale peak being used to identify the onset and end of that exhale.

The individual exhales may, in particular, be identified using an adaptive thresholding method, and the exhale identification algorithm may therefore employ an adaptive thresholding method. The exhale identification algorithm may therefore be an adaptive thresholding algorithm. Adaptive thresholding methods, such as Otsu's adaptive thresholding method, employ a moving window function that is applied to the audio signal envelope to determine an adapted threshold that varies across the envelope. The value of the adaptive threshold is different for, and is adapted to, each portion of the envelope. The moving window function divides the envelope into segments, and the adaptive threshold is determined for each segment of the envelope. The moving window function may be applied to the audio signal envelope in both (i.e. forward and backward) directions and the average (i.e. mean) of the adaptive threshold determined in both directions taken as the adaptive threshold. The adaptive threshold is then used to identify the exhales in the breathing audio data, specifically in the signal envelope. In particular, exhales are identified in the audio signal envelope where the amplitude of the audio signal envelope exceeds the adaptive threshold, and each continuous portion of the audio signal envelope having an amplitude that exceeds the adaptive threshold is identified as an individual exhale. The onset and end (offset) of each exhale may also be identified, namely as the first and last time point in each exhale.

The adapted thresholding method may be adapted based on the estimated respiration rate. The estimated respiration rate, or the expected inter-breath period, may be used as an input parameter to the adaptive thresholding algorithm, or one or more parameters of the adaptive thresholding method may be adapted or optimised based on the estimated respiration rate. The adaptive thresholding method is optimised based on the estimated respiration rate to more accurately identify the exhales.

The length of a moving window function employed by the adaptive thresholding method may be adapted based on the estimated respiration rate. For example, an expected, or average (e.g. mean) inter-breath period may be calculated based on the estimated respiration rate, and the length of the moving window function may be adjusted based on the expected inter-breath period. For example, the length of the moving window function may be adjusted in proportion to, or relative to, the expected inter-breath period. In one particular example, the length of the moving window function may be set equal to 75% of the expected inter-breath period. The degree, or extent, of overlap of adjacent signal segments defined by the moving window function may also be determined based on the estimated respiration rate. In other words, the moving window function may be defined by a length and a degree of overlap, and the length and/or the degree of overlap of the moving window function may be determined based on the estimated respiration rate. The degree of overlap of the moving window function may be adjusted in proportion to, or relative to, the expected inter-breath period. In one particular example, the degree of overlap may be set equal to 75% of the expected inter-breath period.

Separately from the optimisation of the window function employed by the adaptive thresholding algorithm, step 306 may comprise step 328 of refining the identification of the exhales using the estimated respiration rate, optionally in combination with the optimisation of the window function employed by the adaptive thresholding algorithm. In particular, step 328 may comprise using the estimated respiration rate to identify exhales missed by the exhale identification algorithm (e.g. the adaptive thresholding method) and/or to identify and discard spurious exhales identified by the exhale identification algorithm.

For example, step 328 may comprise merging adjacent exhales identified by the exhale identification algorithm if an inter-breath period between them is shorter than a minimum inter-breath period threshold, the minimum inter-breath period threshold determined based on the estimated respiration rate. The minimum inter-breath period threshold may be determined based on the expected inter-breath period. For example, it may be determined relative to, or in proportion to, the expected inter-breath period. The inter-breath period is a measure of the temporal offset between two exhales. The inter-breath period may be calculated, for example, as the period between the onsets of the adjacent exhales. Therefore, in other words, step 328 may comprise merging adjacent exhales identified by the exhale identification algorithm that are offset by an inter-breath period that is shorter than the minimum inter-breath period threshold. The adapted threshold may then be reduced to a value less than the amplitude of the audio data envelope between the two adjacent exhales that are merged together.

Step 328 may comprise searching for missing exhales between adjacent exhales identified by the exhale identification algorithm that are separated, or offset, by an inter-breath period that exceeds a maximum inter-breath period threshold, the maximum inter-breath period threshold determined based on the estimated respiration rate. The maximum inter-breath period threshold may be determined based on the expected inter-breath period. For example, it may be determined relative to, or in proportion to, the expected inter-breath period. Searching for missed exhales between adjacent exhales may comprise reducing the adaptive threshold in the interval between the adjacent exhales and identifying missing exhales using the reduced adaptive threshold. In particular, exhales may be identified in the audio signal where the amplitude of the audio signal envelope exceeds the reduced adaptive threshold, and each continuous portion of the audio signal envelope having an amplitude that exceeds the reduced adaptive threshold is identified as an individual exhale.

Step 328 may comprise discarding individual exhales identified by the exhale identification algorithm that have a duration longer than a maximum exhale duration threshold, the maximum exhale duration threshold determined based on the estimated respiration rate. The maximum exhale duration threshold may be determined based on the expected inter-breath period. For example, it may be determined relative to, or in proportion to, the expected inter-breath period. The adapted threshold may then be increased in the portions of the breathing audio data envelope corresponding to the discarded exhales and exhales may then be searched for in the portions of the signal envelope corresponding to the discarded exhales using the increased adapted threshold. In particular, exhales may be identified in the audio signal where the amplitude of the audio signal envelope exceeds the increased adaptive threshold, and each continuous portion of the audio signal envelope having an amplitude that exceeds the increased adaptive threshold is identified as an individual exhale. This searching step may be performed after all the threshold modifications in Step 328 have been made.

Step 328 may comprise discarding the shorter of each pair of adjacent exhales identified by the exhale identification algorithm that are separated by an inter-exhale interval that is shorter than a minimum inter-exhale interval threshold, the minimum inter-exhale interval threshold determined based on the estimated respiration rate. The minimum inter-exhale interval threshold may be determined based on the expected inter-breath period. For example, it may be determined relative to, or in proportion to, the expected inter-breath period. The inter-exhale interval is the period in between two adjacent exhales, i.e. the time between the end of an exhale and the onset of the following or subsequent exhale. The adaptive threshold may be increase to greater than the amplitude of the signal envelope in the portion of the signal envelope corresponding to the discarded exhale to prevent re-detection when missed peaks are searched for using the modified threshold once all threshold modifications had been made.

Step 328 may comprise discarding exhales identified by the exhale identification algorithm that are shorter than a minimum exhale duration threshold. The adaptive threshold may then be increased to greater than the amplitude of the signal envelope in the portion of the signal envelope corresponding to the removed exhale to prevent re-detection when missed peaks are searched for using the modified threshold once all threshold modifications had been made.

Once all the adaptive threshold modifications in step 328 are performed, step 328 may comprise identifying exhales in the signal envelope using the modified adaptive threshold, thereby eliminating the spurious exhales, which now have an amplitude below the modified adaptive threshold, and potentially identifying missed exhales where the adaptive threshold has been reduced.

The refinement of the exhales using the estimated respiration rate in step 328 improves the accuracy of the exhale identification. In particular, the estimated respiration rate is used to remove or modify physiologically incoherent exhales, and relies on the fact that the exhale duration and inter-breath periods are related to the respiration rate in a real-world setting. The estimated respiration rate is therefore used to inform the initial identification of the exhales by the exhale identification algorithm and to then refine the identification of the exhales to improve the accuracy of exhale identification yet further.

A refined respiration rate may then be determined based on the identified exhales in step 308. For example, the refined respiration rate may be determined based on the number of exhales identified in the breathing audio data, in particular the number of identified exhales per unit time.

Other respiration parameters may be determined based on the identified exhales, such as average exhale duration, inter-breath period variation (e.g. variance or standard deviation), a ratio of average exhale duration to respiration rate, or a ratio of average exhale duration to average full breath duration (i.e. a ratio of average exhale duration to average inter-breath period). Where the word “average” is used here, it is generally used to mean the arithmetic mean, but other averages could also be employed. These exhale-derived respiration parameters provide further useful insight into patient health, and may be a valuable tool to the clinician. For example, the ratio of exhale duration to inter-breath period may be helpful in identifying patients that are deteriorating and in need of medical intervention.

Computer-implemented method 300 may optionally comprise step 330 of classifying the quality of the breathing audio data as either acceptable or unacceptable for use in determining one or more respiration features, for example, for identifying exhales. The signal quality may be classified the using a signal classifier trained by machine learning to classify the quality of breathing audio data as acceptable or unacceptable for use in determining the one or more respiration features. In particular, the signal classifier may be trained by machine learning to classify the quality of breathing audio data as acceptable or unacceptable for identifying exhales in the audio data.

If the quality of the audio data is classified as acceptable, step 306 of identifying exhales in the breathing audio data using the estimated respiration rate is performed. On the other hand, if the signal quality is determined by the signal classifier to be unacceptable, the signal may be discarded in step 332.

If the signal quality is classified as being unacceptable, an instruction that the breathing audio data must be re-recorded may be issued in step 334. A prompt to re-record the breathing audio data may be displayed on a display screen of the device used to record the breathing audio data in response to the issuing of the instruction that the breathing audio data must be re-recorded. Once the breathing audio data has been re-recorded, the new breathing audio data that has been recorded are then acquired and the method begins again at step 302. The new, re-recorded breathing audio data are used as the breathing audio data in the method steps that follow the acquisition of the re-recorded breathing audio data. In other words.

The signal classifier may have been trained by supervised machine learning using a training dataset comprising a plurality of breathing audio data recordings previously classified as being acceptable or unacceptable for identifying exhales. The breathing audio data recordings may have been classified as being acceptable or unacceptable for identifying exhales by at least one person. For example, if the at least one person is unable to annotate the exhales in a recording, the recording is marked as unacceptable. Otherwise, if the at least one person is able to annotate the exhales in a recording the recording is classified as acceptable. The signal classifier may classify the quality of the breathing audio data based on a plurality of features, for example statistical features, extracted from the breathing audio data, and may for example employ an ensemble of trees. The signal classifier may therefore be an ensemble of trees classifier that employs a plurality of features extracted from the breathing audio data to classify the quality of the audio data signal. In particular, the signal classifier may employ the estimated respiration rate to determine whether the quality of the breathing audio data is acceptable or unacceptable for identifying the exhales, and the estimated respiration rate may therefore be one of the plurality of features employed by the signal classifier. Employing the estimated respiration rate to classify the signal quality provides an improved measure of the quality of the signal. This is because the estimated respiration rate is a physiologically meaningful parameter, and its value is therefore related to the quality of the signal.

The machine learning algorithm used to train the signal classifier may employ a feature selection in order to reduce the risk of overfitting. For example, an initial plurality of features may be input into the machine learning algorithm, and the feature selection routine may remove features that are highly correlated, e.g. correlated by more than a correlation threshold vale, with other features, for example, as determined using a correlation coefficient such as Spearman's rank order correlation coefficient. The feature selection routine may also perform sequential backward feature selection.

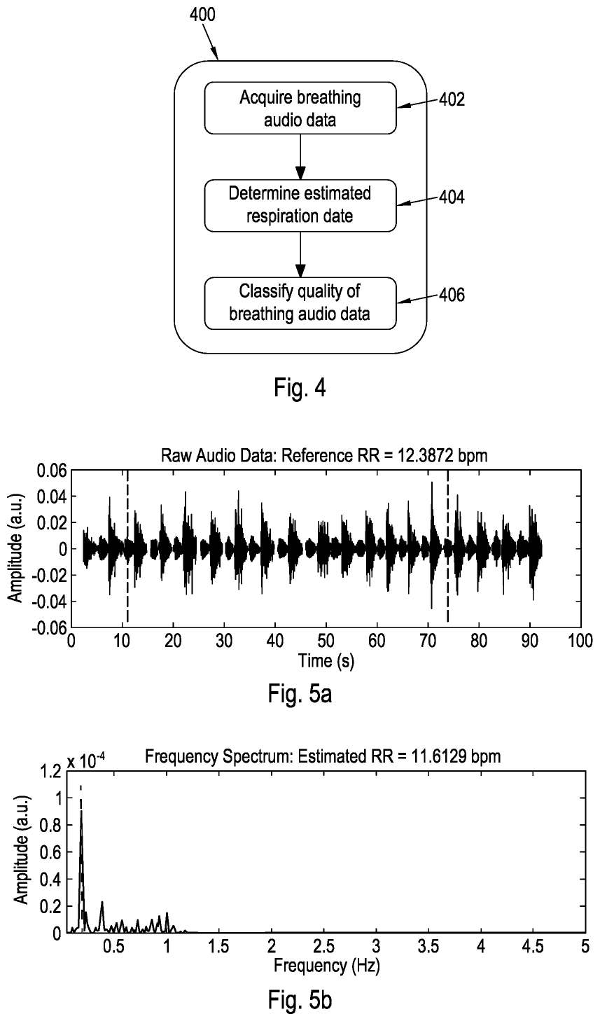

Another computer-implemented method 400 in accordance with the invention is shown in FIG. 4. Method 400 is a computer-implemented method for classifying the quality of breathing audio data as acceptable or unacceptable for use in determining one or more respiration features, such as exhales or a respiration rate. Method 400 comprises step 402 of acquiring breathing audio data. Method 400 may then optionally comprise step 404 of determining an estimated respiration rate based on the breathing audio data. Step 404 may be performed as per step 304 described in relation to method 300. Method 400 may then comprise step 406 of classifying the quality of the audio data as acceptable or unacceptable for determining the one or more respiration features using a signal classifier trained by machine learning to classify the quality of breathing audio data as acceptable or unacceptable for determining the one or more respiration features. Step 406 may be performed as per step 330 in method 300. The signal classifier employed in step 406 may employ the estimated respiration rate optionally determined in step 304 to determine whether the quality of the breathing audio data is acceptable or unacceptable for determining the one or more respiration features.

Method 400 also comprise, if the quality of the breathing audio data is classified as acceptable, determining the one or more respiration features based on the breathing audio data, and may therefore comprise further steps corresponding to steps 306 and optionally also step 308 in method 300. If an estimated respiration rate is first determined, then the step corresponding to step 308, if performed, comprises determining a refined respiration rate, whereas if no estimated respiration rate has previously been determined this step may instead be said to comprise determining a respiration rate (rather than a refined respiration rate). If these further steps are performed method 400 may instead by a method for characterising breathing audio data, for identifying exhales and/or for determining a respiration rate. In general, method 400 may further comprise any or all of the steps of method 300 in addition to those described above.

### Example

The example described below is intended to illustrate various aspects of the invention, and is not to be considered as limiting on the scope of the invention. The example combines various optional features of the invention, and it should be understood that the invention can be performed using other implementations that differ from the example described below, for example by omitting one or more optional features or steps.

### Data Collection

Subjects (individuals) held their phone horizontally with the microphone 2 cm from their mouth/nose, while sitting comfortably in a quiet location, and recorded their natural breathing for 90 seconds. The audio data recordings were sent by email to researchers. Subjects completed a questionnaire to provide information on demographics, phone technical specifications, COVID-19 symptoms and diagnosis.

Two researchers independently annotated inhales and exhales for each recording. The onset and end of each inhale and exhale were defined as the mean of the independent annotations. If it was not possible to annotate exhales during a recording, the recording was labelled as being of unacceptable signal quality. The averaged annotations were used as reference measures to assess the performance of the respiration monitoring algorithm.

### Dataset

210 subjects submitted a dataset of 217 recordings. Two subjects participated as control subjects, and also as COVID-19 subjects at a later date. Four control subjects submitted two recordings, one control subject submitted three recordings, and one COVID-19 subject submitted two recordings. In COVID-19 subjects, days since diagnosis was used to define the time since disease onset, if available. If diagnosis date was not reported, the date of symptom onset was used.

It was possible to annotate both inhales and exhales in 154 recordings, and only exhales in a further 42 recordings. Four of these recordings were excluded due to differences between researcher annotations, resulting in a total of 192 recordings (88.5%) classified by the researchers as having acceptable signal quality.

Of the 192 acceptable recordings, 140 recordings were submitted by 144 control subjects and 48 recordings were submitted by 46 subjects who had been diagnosed with COVID-19. The COVID-19 subjects were categorised based on days since diagnosis of COVID-19 and the presence of symptoms. If a diagnosis date was not reported, the date of symptom onset was used. 20 recordings were submitted by COVID-19 subjects with a recent diagnosis (day 1-21) and 28 recordings from subjects who were diagnosed more than 21 days prior to participating in the study. Twenty-four recordings were submitted by COVID-19 subjects who were not experiencing symptoms, while 24 recordings were submitted by COVID-19 subjects who were experiencing symptoms.

### Pre-Processing

Audio data for stereo recordings (31 recordings) were first averaged. Signals were then filtered using an 8th order Butterworth low-pass filter with a 1 kHz cut-off frequency. To reduce signal artefacts at the start and end of the recordings, approximately 60 seconds of data were selected for signal quality classification and initial respiration rate estimation. The start of the analysis segment was defined as midway between the exhale onset immediately prior to 10 seconds and the subsequent exhale onset. The end of the analysis segment was defined as the midpoint between the first and second exhale onsets at least 60 seconds after the start of the analysis segment. FIG. 5a shows an example of a breathing audio data recording, with the data used for signal quality classification and initial respiration rate estimation bounded by the two vertical dashed lines.

### Respiration Rate Estimation

An initial respiration rate estimate was obtained based on the fundamental frequency of the respiration signal envelope as determined from the harmonic product spectrum. A 50 Hz notch filter was implemented if the power at 50 Hz was greater than 20% of the total power in the audio signal. Signals were then rectified and low-pass filtered using a 4th order Butterworth filter with a cut-off frequency of 1 Hz to obtain an envelope of the respiration data.

The power spectrum of the envelope was then estimated using Welch's method with a Hamming window of adaptive length and 50% overlap. The Hamming window length was 60 seconds as standard (i.e. for audio data recordings determined to not contain unusual or anomalous features), or 30 seconds for recordings containing unusual breathing patterns or artefacts (caused, for example, by coughing, sighing, yawning, or a change in phone position). To identify anomalous features, a moving 2 second RMS (root mean square) window with 50% overlap was applied to the signal envelope, and the peaks were detected. If the maximum peak in the moving RMS signal was more than five times the median peak amplitude, the recording was deemed to contain anomalous features, and the Hamming window length was set to 30 seconds (18.89% of signals were processed using a 30 second window). Any large peaks (i.e. those having an amplitude more than five times the median peak amplitude) were then rescaled to the amplitude of the median amplitude of all peaks identified in the audio data. A power spectrum calculated based on the raw audio data shown in FIG. 5a is shown in FIG. 5b, with the peak corresponding to the fundamental frequency identified by the vertical dashed line.

The respiration rate was estimated as the fundamental frequency of the respiration signal envelope. To assist in selecting the fundamental frequency, rather than a harmonic, the harmonic product spectrum of the audio signal envelope was calculated based on the power spectrum, and the first peak in the harmonic product spectrum having a frequency of greater than 0.09 Hz (corresponding to a minimum acceptable respiration rate of 5.5 bpm) and an amplitude of at least 80% of the maximum amplitude in the harmonic product spectrum was selected. The reciprocal of this initial respiration rate estimate (estRR) was used to calculate the expected inter-breath period (IBP).

### Signal Quality Classification

An ensemble of trees classifier, Extreme Gradient Boost (XGBoost) using an exact greedy strategy, was developed to classify the 217 recordings as acceptable or unacceptable, based on researcher annotations classifying each recording as either acceptable or unacceptable.

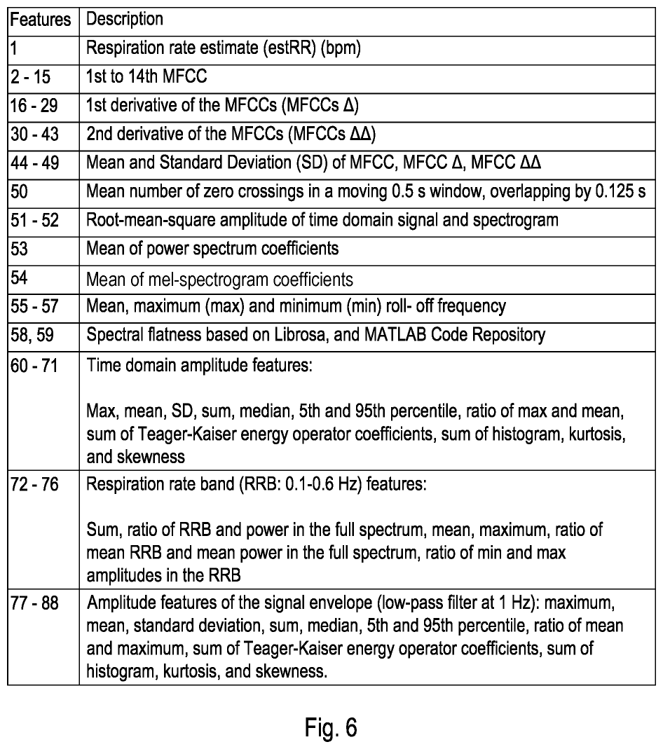

Eighty-eight time-domain and frequency-domain features were extracted from each recording, as summarised in FIG. 6. The feature set included the initial respiration rate estimate (estRR), and a range of time and frequency domain features, including the Mel-Frequency Cepstral Coefficients (MFCC) and its variants, including MFCC Delta and MFCC Delta-Delta.

Classifier performance was assessed within leave-one-out (LOO) cross-validation. The training dataset within each LOO fold was balanced by over-sampling the unacceptable recordings using Borderline Synthetic Minority Over-sampling Technique (SMOTE). A feature selection routine was implemented to reduce the risk of overfitting. First, features were eliminated if they were highly correlated with other extracted features, i.e. when Spearman's rank order correlation coefficient (Rho) between the features exceeding 0.8. Second, sequential backward feature selection was applied to the remaining features, with a greedy strategy optimizing the area under the Receiver Operating Characteristic Curve (ROC AUC) within 5-fold cross-validation. The selected features were used to train a XGBoost classifier within each fold. Bayesian optimization was used to optimize the classifier parameters.

Classifier performance was assessed using the test data for each fold, with mean performance metrics calculated across all LOO folds. The ROC AUC was calculated, along with the mean accuracy, recall, precision, specificity and F1 score across all cross-validation folds calculated.

### Exhale Detection and Respiration Rate Refinement

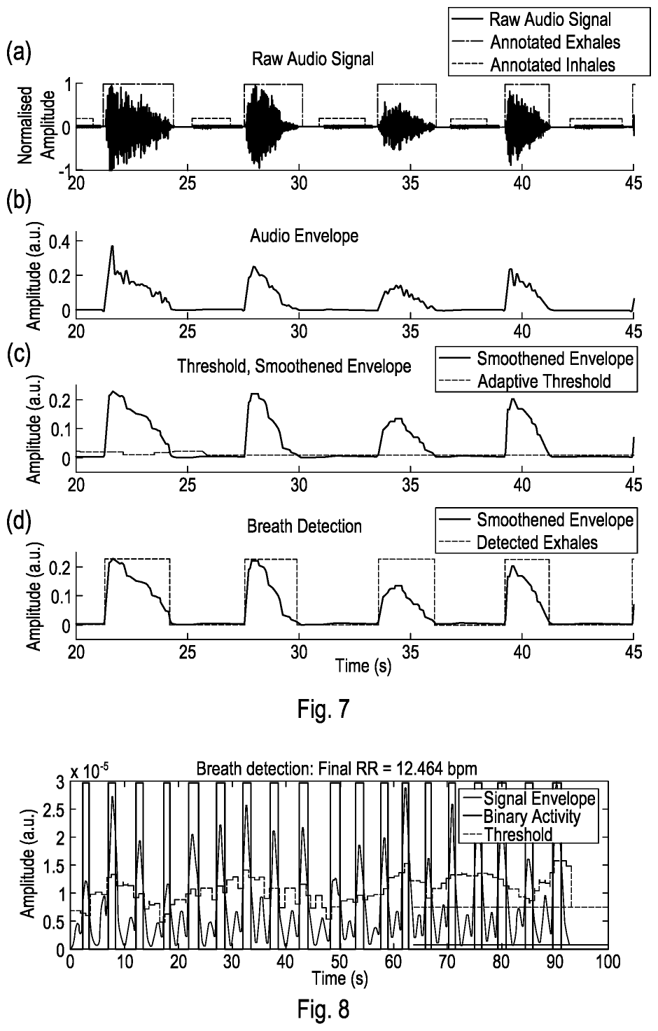

A method to identify the onset and end of each exhalation during the full 90 second respiration recording was then developed. An 8th order Butterworth low-pass filter with a cut-off frequency of 5 Hz was applied to the rectified audio data. The resulting signal envelope was then smoothed using a 0.5 s moving median filter, with a step size of 1 sample. FIG. 7a shows an example raw audio data segment, FIG. 7b shows the effect on the signal of rectification and low-pass filtering to generate an audio signal envelope.

Otsu's adaptive image thresholding method was applied to the smoothed envelope to detect exhalations in the respiration signal. FIG. 7c shows a smoothed envelope with the adaptive threshold overlaid as the dashed line. To account for changes in amplitude during the recording, the thresholding method used a window length equal to 75% of the expected IBP, with an overlap of 75%. This thresholding method was performed on the signal envelope in both the forward and backwards directions and the mean of the threshold calculated in both directions was calculated and used as the adaptive threshold to reduce the influence of isolated large amplitude exhalations. Exhalations were detected if the amplitude of the smoothened signal envelope was greater than the adaptive threshold, as illustrated in FIG. 7d. FIG. 8 shows another breathing audio data recording with the smoothed signal envelope, the adapted threshold, and the exhales (identified by the peaks in the binary square-wave) identified.

Missed or spurious exhales where then searched for and identified using the IBP. If the inter-breath period between two successive exhales was less than 50% of the expected IBP the exhales were merged and the threshold within this interval was reduced to 99% of the signal envelope amplitude to prevent the error from recurring when missed peaks were searched for using the modified threshold once all threshold modifications had been made. Similarly, if the inter-breath period between two successive exhales was greater than 150% of the expected IBP, the adaptive threshold in the respective inter-breath period was reduced using a Hamming window with a length equal to the respective IBP and a peak amplitude of half of the threshold in the respective IBP. The interval was searched for a potentially missed exhale using the reduced adaptive threshold once all threshold modifications had been made.

Given that the typical inhale to exhale ratio during resting respiration is 1:2, a minimum exhale duration was determined as 0.2 seconds, as even with a high respiration rate of 60 bpm, an exhale would be expected to last approximately 0.67 s. Exhales were removed if their duration was less than the minimum duration, with the threshold increased to 1% greater than the amplitude of the envelope in the window corresponding to the removed exhale to prevent re-detection when missed peaks were searched for using the modified threshold once all threshold modifications had been made.

Similarly, exhales with duration greater than 60% of the expected IBP were considered too long, and the adaptive threshold was increased by 50% to shorten the exhale duration.

Finally, if the offset of an exhale preceded the onset of the following exhale by less than 40% of the expected IBP, the shorter of the two exhales was removed and the threshold was increased to 1% greater than the amplitude of the envelope in that window.

Once all the adaptive threshold modifications had been performed, exhales were again identified using the modified adaptive threshold.

### Statistical Analysis

Respiration rate, mean exhale duration, and the ratio of mean exhale duration to mean inter-breath period (normalized exhale duration) were computed for each audio data recording. The mean absolute error (MAE) was used to assess the accuracy of the estimated respiration rate and exhale durations compared with the researcher annotations.

Each derived feature (respiration rate, exhale duration and normalized exhale duration) was compared for each sub-group of the cohort using one-way ANOVA, with pairwise Tukey-HSD tests used to account for multiple comparisons. Firstly, control subjects were compared with COVID day 1-21 and COVID day >21 groups, and secondly control subjects were compared with COVID symptoms and COVID no symptoms groups.

To account for the different subject numbers in each group, age and gender-matched datasets were used for statistical analysis, with subjects in the control and COVID day >21 groups selected to match the age and gender distribution of the COVID day 1-21 group.

Signal processing, classifier development, and statistical analyses were performed using Matlab (The Mathworks, Natick, MA, USA) and python. The python libraries Librosa, Scikit-learn and SciPy were used. p-values less than 0.05 were considered statistically significant.

### Signal Quality Classification Results

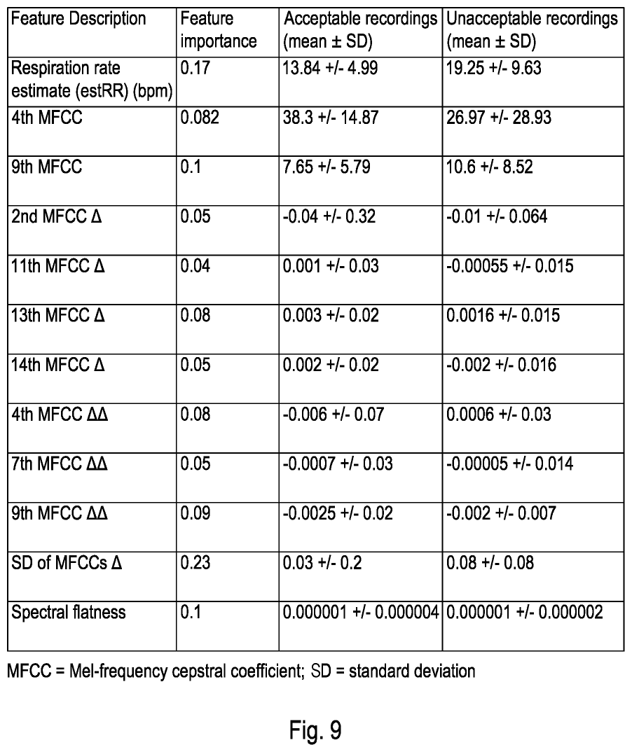

Twelve features were selected for the final signal quality classifier model, as detailed in FIG. 9. The initial respiration rate estimate (estRR) was one of the features selected. The cross-validated classifier accuracy for classifying the recordings as acceptable or unacceptable in line with the researcher annotations was 88%, and the precision, recall and F1 score were all 93.2%. The ROC AUC was 0.83.

### Respiration Analysis Results

Respiration rate was estimated with a MAE of 0.79±2.44 bpm (7.14±25.33%), and median error 0.07 bpm, compared with the researcher annotations. Respiration rate MAE was 0.73±2.37 (7.44±28.47%) for recordings from control subjects, 1.13±3.21 bpm (6.06±12.76%) for recordings from COVID-19 (day >21) subjects, and 0.84±1.35 bpm (6.52±9.54%) for recordings by COVID-19 (day 1-21) subjects. Error in respiration rate was less than 1 bpm for 87.5% of recordings, while 92.71% of files were accurate within 2 bpm.

The MAE for exhale duration for all acceptable recordings was 0.21±0.23 s (14.55±24.15%), with a median error of 0.14 s. The MAE for exhale duration was 0.21±0.19 s (12.77±13.01%) in the control group, 0.24±0.32 s (25.16±53.36%) in the COVID-19 (day >21) recordings, and 0.20±0.26 s (12.53±14.56%) in the COVID-19 (day 1-21) recordings. There was no significant difference between the MAE for respiration rate or exhale duration between the different groups.

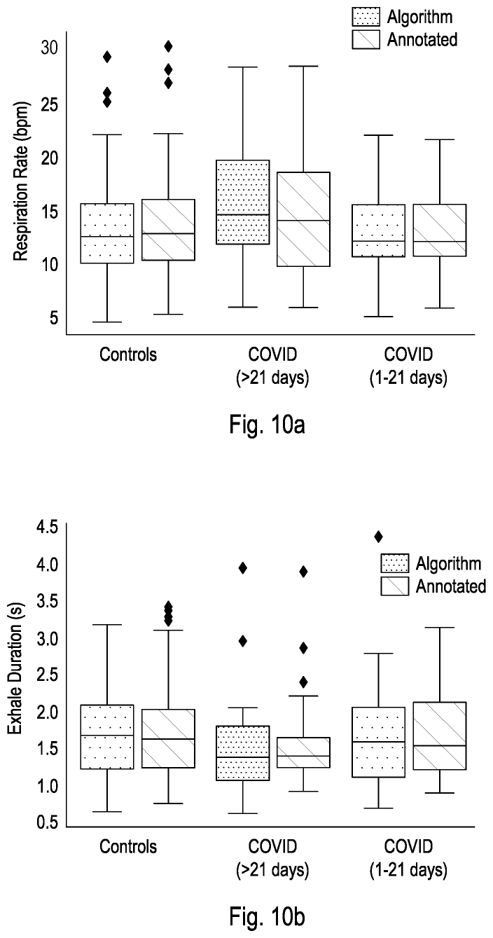

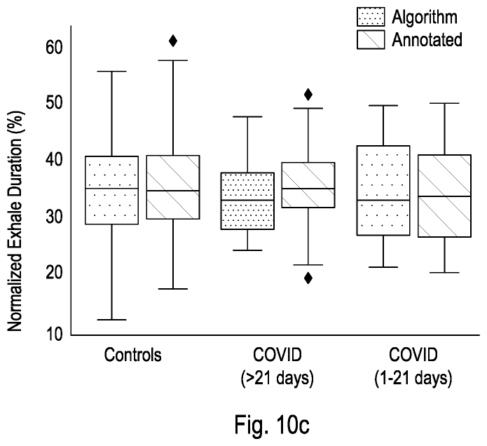

The coefficient of determination, R², was 0.72 for respiration rate and 0.76 for exhale duration. The intra-class correlation coefficient, r, was 0.92 for respiration rate, and 0.93 for exhale duration, indicating excellent agreement. FIGS. 10a-c illustrate graphically the close agreement between the respiration rate (FIG. 10a), exhale duration (FIG. 10b) and normalized exhale duration (FIG. 10c) calculated using the algorithm (filled boxes, left) and from researcher annotations (hatched boxes, right) for all groups, demonstrating the robustness and accuracy of the algorithm in calculating vital respiration parameters for both healthy subjects and those suffering from respiratory distress and exhibiting abnormal breathing patterns.

The results therefore demonstrate that the method of the invention provides an accurate and reliable means for determining respiration parameters, such as respiration rate and exhale duration, using breathing audio data that has been recorded remotely using personal smart devices. The signal classifier is also effective at classifying quality of audio recordings as being either acceptable or unacceptable for use in determining such respiration parameters. The results also demonstrate that the methods of the invention are equally effective at determining respiration parameters of those with and without an underlying respiratory condition, in this case COVID-19, which can give rise to abnormal breathing patterns that often cause problems for algorithms.

It will be appreciated by those skilled in the art that while the invention has been described above in connection with particular embodiments and examples, the invention is not necessarily so limited, and that numerous other embodiments, examples, uses, modifications and departures from the embodiments, examples and uses are intended to be encompassed by the claims attached hereto.

Although the appended claims are directed to particular combinations of features, it should be understood that the scope of the disclosure of the present invention also includes any novel feature or any novel combination of features disclosed herein either explicitly or implicitly or any generalisation thereof, whether or not it relates to the same invention as presently claimed in any claim and whether or not it mitigates any or all of the same technical problems as does the present invention.

Features which are described in the context of separate embodiments may also be provided in combination in a single embodiment. Conversely, various features which are, for brevity, described in the context of a single embodiment, may also be provided separately or in any suitable sub-combination. The applicant hereby gives notice that new claims may be formulated to such features and/or combinations of such features during the prosecution of the present application or of any further application derived therefrom. Methods, systems, computer readable media and computer programs may each have corresponding features definable and/or combinable with respect to each other, and these embodiments are specifically envisaged.

For the sake of completeness, it is also stated that the term “comprising” does not exclude other elements or steps, the term “a” or “an” does not exclude a plurality, a single processor or other unit may fulfil the functions of several means recited in the claims and any reference signs in the claims shall not be construed as limiting the scope of the claims.

1. A computer-implemented method for characterising breathing audio data, the method comprising:
acquiring breathing audio data;
determining an estimated respiration rate based on the breathing audio data; and
identifying exhales in the breathing audio data using the estimated respiration rate.

2. The method of claim 1, further comprising determining a refined respiration rate based on the identified exhales.

3. The method of claim 1, wherein determining the estimated respiration rate comprises performing a spectral analysis of the breathing audio data to determine the estimated respiration rate.

4. The method of claim 1, wherein determining the estimated respiration rate comprises calculating a frequency spectrum of the breathing audio data, and determining the estimated respiration rate based on the frequency spectrum, optionally wherein the frequency spectrum is a power spectrum.

5. The method of claim 4, further comprising determining a fundamental frequency of the breathing audio data based on the frequency spectrum, wherein the estimated respiration rate is determined based on the fundamental frequency.

6. The method of claim 5, further comprising calculating a harmonic product spectrum of the breathing audio data based on the frequency spectrum, and identifying the fundamental frequency based on the harmonic product spectrum.

7. The method of claim 4, wherein the frequency spectrum is determined using a window function having an adaptable length, and wherein calculating the frequency spectrum comprises determining whether the breathing audio data contains anomalous features, and adapting the length of the window function based on whether the breathing audio data is determined to contain anomalous features,
optionally wherein the length of the window function is adapted to a first length if the breathing audio data is determined to not contain anomalous features, or to a second length if the breathing audio data is determined to contain anomalous features, wherein the second length is shorter than the first length.

8. The method of claim 7, wherein the breathing audio data is determined to contain anomalous features if one or more large peaks having an amplitude exceeding a threshold amplitude is identified in the breathing audio data,
optionally wherein the large peaks are rescaled to reduce their amplitude in the breathing audio data prior to calculating the frequency spectrum.

9. The method of claim 1, wherein identifying the exhales in the breathing audio data using the estimated respiration rate comprises identifying exhales in the breathing audio data using an exhale identification algorithm adapted based on the estimated respiration rate; and optionally or preferably; wherein the exhale identification algorithm employs an adaptive thresholding method that is adapted based on the estimated respiration rate; and, optionally or preferably, wherein the length of a moving window function employed by the adaptive thresholding method to determine an adaptive threshold used to identify the exhales is adapted based on the estimated respirate rate, optionally wherein a degree of overlap of the moving window function is adapted based on the estimated respiration rate.

10. (canceled)

11. (canceled)

12. The method of claim 9, wherein the estimated respiration rate is used to identify exhales missed by the exhale identification algorithm and/or to identify spurious exhales identified by the exhale identification algorithm.

13. The method of claim 9, wherein adjacent exhales identified by the exhale identification algorithm are merged if an inter-breath period between them is shorter than a minimum inter-breath period threshold, wherein the minimum inter-breath period threshold is determined based on the estimated respiration rate.

14. The method of claim 9, wherein missed exhales are searched for between adjacent exhales identified by the exhale identification algorithm that are separated by an inter-breath period that exceeds a maximum inter-breath period threshold, wherein the maximum inter-breath period threshold is determined based on the estimated respiration rate.

15. The method of claim 9, wherein exhales identified by the exhale identification algorithm having a duration longer than a maximum exhale duration threshold are discarded, wherein the maximum exhale duration threshold is determined based on the estimated respiration rate; or wherein, if the interval separating adjacent exhales is shorter than a minimum interval threshold, the shorter of the adjacent exhales is discarded, wherein the minimum interval threshold is determined based on the estimated respiration rate.

16. (canceled)

17. The method of claim 1, further comprising classifying the quality of the breathing audio data as acceptable or unacceptable for identifying exhales using a signal classifier trained by machine learning to classify the quality of breathing audio data as acceptable or unacceptable for identifying exhales, and performing the steps of identifying exhales in the breathing audio data using the estimated respiration rate and determining a refined respiration rate based on the identified exhales if the quality of the audio data is classified as acceptable.

18. The method of claim 17, further comprising, if the quality of the audio data is classified as unacceptable, issuing an instruction that the breathing audio data must be re-recorded, and acquiring re-recorded breathing audio data; and/or wherein the signal classifier has been trained using a training dataset comprising a plurality of breathing audio data recordings previously classified as being acceptable or unacceptable for identifying exhales; and/or wherein the signal classifier employs the estimated respiration rate to determine whether the quality of the breathing audio data is acceptable or unacceptable for identifying the exhales.

19. (canceled)

20. (canceled)

21. A computer system for characterising breathing audio data, wherein the computer system is configured to:
acquire breathing audio data;
determine an estimated respiration rate based on the breathing audio data; and
identify exhales in the breathing audio data using the estimated respiration rate.

22. One or more computer-readable storage media comprising instructions that, when executed by a computer, cause the computer to:
determine an estimated respiration rate based on breathing audio data; and
identify exhales in the breathing audio data using the estimated respiration rate.

23. A computer-implemented method for classifying the quality of breathing audio data as acceptable or unacceptable for use in determining or identifying one or more respiration features, the method comprising:
acquiring breathing audio data;
classifying the quality of the breathing audio data as acceptable or unacceptable for determining the one or more respiration features using a signal classifier trained by machine learning to classify the quality of breathing audio data as acceptable or unacceptable for determining or identifying the one or more respiration features.

24. A computer system for classifying the quality of breathing audio data as acceptable or unacceptable for use in determining or identifying one or more respiration features, the computer system comprising:
a processor configured to classify the quality of the audio data as acceptable or unacceptable for determining the one or more respiration features using a signal classifier trained by machine learning to classify the quality of breathing audio data as acceptable or unacceptable for determining or identifying the one or more respiration features.

25. One or more computer-readable storage media comprising instructions that, when executed by a computer, cause the computer to:
classify the quality of breathing audio data as acceptable or unacceptable for determining or identifying one or more respiration features using a signal classifier trained by machine learning to classify the quality of breathing audio data as acceptable or unacceptable for determining or identifying the one or more respiration features.
